(19) United States
(12) Patent Application Publication
(10) Pub. No.: US 2010/0145050 A1
(43) Pub. Date: Jun. 10, 2010

(54) ORTHOGONALLY FIXED COMPOUNDS FOR ELECTROOPTICAL APPLICATIONS

(75) Inventors: Hans-Hermann Johannes, Braunschweig (DE); Wolfgang Kowalsky, Braunschweing (DE); Martin Dümeland, Halle (DE); Ulfla Wrenz, Ludwigshafen (DE)

Correspondence Address: GREER, BURNS & CRAIN, 300 S WACKER DR, 25TH FLOOR, CHICAGO, IL, 60606, US

(73) Assignee: TECHNISCHE UNIVERSITAT BRAUNSCHWEIG, Braunschweig (DE)

(21) Appl. No.: 11/794,518

(22) PCT Filed: Jan. 10, 2005
(86) PCT No.: PCT/EP2005/050086

§ 371 Date: Feb. 24, 2010

(51) Int. Cl. C07D 471/06; C07C 211/61; C07D 495/10; C07D 413/14; C07D 409/14; C07D 455/04

(52) U.S. Cl. 546/15; 564/427; 548/407; 548/216; 548/145; 549/43

(57) ABSTRACT

The present invention relates to chemical compounds that can be used in electrooptical applications. The electrically conductive and optical properties of these compounds, e.g. hole transporting, electron transporting and/or light emitting properties can be predetermined by substituting the core structure of these compounds with respective residues. The core structure (1) of the compounds according to the invention comprises two opposing aromatic moieties which are chemically bonded through an intermediate central atom. This central atom has a tetraedric configuration and therefore provides an orthogonal orientation to the organic moieties.

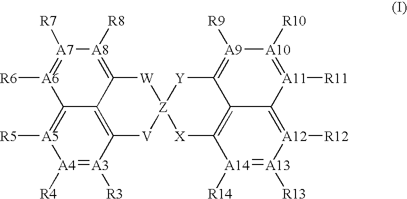

The present invention relates to chemical compounds that can be used in electrochemical or electrooptical applications. The electric and/or optical properties of these compounds, e.g. hole transporting, electron transporting, hole injecting, electron injecting and/or light emitting properties can be predetermined by substituting the core structure of these compounds with respective residues. Electric and electrooptic applications comprise organic light emitting diodes (OLEDs), organic field effect transistors (OFETs), lasers, and photovoltaic devices suitable for photovoltaic solar energy conversion.

The core structure of the compounds according to the invention comprises two opposing aromatic moieties which are chemically bonded through an intermediate central atom having a tetraedric configuration to provide an orthogonal orientation to the bonded aromatic moieties.

### STATE OF THE ART

WO 96/17035 discloses heterospiro compounds and their use as electroluminescent materials, generally formed of two conjugated systems which are directly linked by a central atom, for example silicon, germanium or tin. Furthermore, there is disclosed a heterospiro compound having two biphenyl groups as symmetrical aromatic moieties which are linked via the central tetraedric atom to form a spiro compound. The biphenyl groups are linked to one another by the intermediate central atom, each biphenyl group forming a fluorene structure with its two phenyl moieties and the central atom.

EP 0676461 A2 discloses compounds analogous to WO 96/17035, wherein the central atom is a carbon atom.

### OBJECTS OF THE INVENTION

It is an object of the invention to provide compounds which are an alternative to known spiro compounds. It is preferred that the alternative compounds have a high glass transition temperature and good long term stability. Further, it is preferred that the alternative compounds can be derivatized to introduce the electrical and/or luminescent properties desired for electrooptical applications.

### GENERAL DESCRIPTION OF THE INVENTION

The present invention achieves the above-mentioned object by providing the core structure of general formula I for compounds suitable for electrooptical and/or electroluminescent applications:

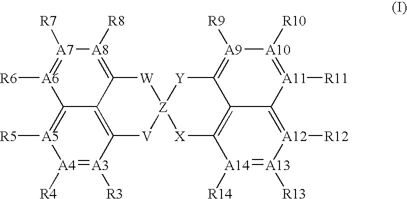

wherein V, W, X and Y can be selected from at least divalent atoms or groups, e.g. —S—, —NR—, —O—,

a carbonyl group, —SO₂—, and di-substituted silicon, —CRR—, and a chemical bond, with R any (hetero-) alkyl or (hetero-) aryl or hydrogen, wherein at least one of V, W and X, Y, respectively are no chemical bonds but atoms,

wherein R3 to R14 are independently selected from (hetero-) alkyls, (hetero-) aryls, —NR′₂, —OR′, —SR′, —CN, —F, —CF₃, with R′ independently selected from substituted or unsubstituted (hetero-)alkyl, (hetero-)aryl or hydrogen, and electrooptically functional groups, wherein two or more of R3 to R14 can be condensed arenyl groups or groups forming a higher condensed derivative of the core structure of formula I,

wherein A3 to A14 each are independently a carbon or nitrogen atom, and

wherein Z is a central tetraedric atom.

The central atom Z may be formed by silicon, germanium and, preferably, carbon.

Core structure I comprises a first and a second condensed aromatic system which are connected via central atom Z. Preferably, the first and second condensed aromatic systems are arranged at central atom Z in a position essentially opposite one another. The condensed aromatic systems are each linked to the central atom Z through their adjacent α, α′ carbon atoms and by intermediate residues V, W, arranged between the α carbon atoms of the first condensed aromatic system and central atom Z and intermediate residues X, Y, respectively, arranged between the α′ carbon atoms of the second condensed aromatic system and central atom Z.

Accordingly, the linkage of the first and second condensed aromatic systems to central atom Z is independently formed as a four-, five- or six-membered ring including the α carbon atoms or the α′ carbon atoms, which are part of the first condensed aromatic system and of the second condensed aromatic system, respectively.

In one embodiment, one of intermediate residues V, W linking the α carbon atoms of the first condensed aromatic system to central atom Z is an atom, whereas the other intermediate residue W, V, respectively, is a chemical bond, directly linking one of both α carbon atoms to the central atom Z, forming a five-membered ring which comprises central atom Z, one intermediate residue and the α carbon atoms of the first condensed aromatic system. In a first alternative embodiment, both intermediate residues V, W are atoms, same or different, each arranged between one of both α carbon atoms of the first condensed aromatic system and central atom Z, forming a six-membered ring comprising the α carbon atoms of the first condensed aromatic system, both intermediate residues and central atom Z. In a second alternative embodiment, both V and W are single bonds, forming a four-membered ring, directly linking central atom Z to both α carbon atoms.

Independent from the embodiment of the linkage of the first condensed aromatic system to the central atom, the second condensed aromatic system is linked to the central atom with at least one intermediate residue X, Y being an at least divalent atom or residual group. In one embodiment, the second condensed aromatic system is linked to central atom Z through its α′ carbon atoms with one of intermediate residues X, Y being an atom and the other one of Y, X, respectively, being a chemical bond, forming a five-membered ring between the condensed aromatic system and central atom Z including either intermediate residue X or Y. In an alternative embodiment, both intermediate residues X, Y, respectively are atoms, each arranged between one of the α′ carbon atoms of the second condensed aromatic system and the central atom Z, forming a six-membered ring.

In a preferred embodiment, one of or both of intermediate residues V, W, and X, Y, respectively, are di-substituted carbon atoms, preferably methylene groups. Alternatively, one of V, W and X, Y, respectively, is a di-substituted carbon atom, preferably CR1R2, whereas the other intermediate residue is sulfur, oxygen or a non-substituted or mono-substituted nitrogen.

The bonds between each of the condensed aromatic systems and the central atom are non-conjugated bonds, providing for electronic isolation of the first and second condensed aromatic systems. The respective substituents can be linked conjugatedly or non-conjugatedly to their respective condensed aromatic systems.

The condensed aromatic systems of the core structure may form part of higher anellated aromatic moieties, for example a naphthyl moiety that provides α and α′carbon atoms for linkage to central atom Z may be comprised in an anthracene moiety, a naphthacene or a pentacene moiety as well as in a phenanthrene, chrysene, acenaphthylene, pyrene, coronene, benzo(a)pyrene, or naphthopyrene moiety or heteroatom substituted homologs thereof, preferably providing carbon atoms in positions α and α′. However, positions α and α′ can also be filled by heteroatoms, e.g. Si, or Ge.

The central structure according to general formula I provides the compounds according to the invention with the advantageous properties of having a low propensity to crystallize, which is reflected in a high glass transition temperature. High glass transition temperatures are desired for compounds according to the invention in electrical, especially in electrooptical applications. It is assumed that the steric confirmation of the central structure, arranging the first and second condensed aromatic systems in an orthogonally orientated position is the cause for the advantageous properties of compounds according to the invention.

Substituent groups R3 through R14 can be electrooptically functional groups like hole injecting moieties or hole transporting moieties, electron injecting moieties or electron transporting moieties, and/or luminescence emitting moieties, or non-EL groups like (hetero-) alkyl and (hetero-) aryl groups unless they represent higher aromatic substituents which in combination with the respective condensed aromatic system form higher anellated systems like anthracene, naphthacene, pentacene, phenanthrene, chrysene, acenaphthylene, pyrene, coronene, benzo(a)pyrene, naphthopyrene or condensates thereof. However, at least one substituent having EL properties is linked to a condensed aromatic system, the EL functions provided in each condensed aromatic system may be selected independently.

EL functional group residues conferring hole transporting characteristics onto the core structure of general formula I can be selected from tris-[(N,N-diaryl)amino]-triphenylamines like 4,4′,4″-tris[(N-(1-naphthyl)-N-phenyl-amino-triphenylamine](1-TNATA) and its derivatives, 4,4′,4″-tris[(N-(2-naphthyl)-N-phenyl amino)-triphenylamine](2-TNATA) or 4,4′,4″-tris[(N-(3-methylphenyl)-N-phenyl-amino)-triphenylamine] (m-TDATA) and its derivatives, 4,4′,4″-tris(carbazole-9-yl)triphenylamines; N,N,N′,N″-tetraarylbenzidines as N,N,N′,N′-tetraphenylbenzidine and its derivatives, N,N′-bis(1-naphthyl)-N,N′-diphenyl-benzidine (α-NPD), N,N′-di(naphthalene-2-yl)-N,N′-dipbenylbenzidine (β-NPD), 4,4′-bis(carbazole-9-yl)biphenyl (CBP) and its derivatives, and their heteroatom substituted analogs (e.g. thienyl-, selenyl-, furanyl-derivatives); 4,4′-bis(2,2′-diphenylvinyl)-1,1′-biphenyl (DPVBI); triarylamines and their derivatives, 4,4′-bis(N,N-diarylamino)-terphenyls, 4,4′-bis(N,N-diarylamino)-quarterphenyls and their homologs and derivatives.

EL functional group residues conferring electron transporting characteristics onto the core structure of general formula I can be selected from 4,7-diphenyl-1,10-phenanthroline (Bphen) and derivatives thereof as 2,9-dimethyl-4,7-diphenyl-1,10-phenanthroline (BCP), 2,5-diaryloxadiazoles and derivatives thereof as 2-(p-tert.-butylpheny1)-5-(p-biphenyl)-oxadiazole (PBD), oligo-(benzoxadiazol-2yl)-arenes and derivatives thereof as bis-2,5-(5-tert.-butyl-benzoxadiazol-2-yl)-thiophene (BBOT), 1,3-bis[5-(aryl)-1,3,4-oxadiazol-2yl]benzenes and derivatives thereof as 1,3-bis[5-(p-tert.-butylphenyl)-1,3,4-oxadiazol-2yl]benzene (OXD-7), 2,5-diaryltriazoles and derivatives like 2-(p-tert.-butylphenyl)-5-(p-biphenyl)-triazole (TAZ).

EL functional group residues conferring emitter characteristics onto the core structure of general formula I can be selected from residues which in combination with the condensed aromatic system result in a dye. Dyes resulting from this combination may for example be coumarins, rhodamines, merocyanines, e.g. derivatives of DCM, DCM2, cyanines or oxonoles.

In the alternative to the one or more EL functional group residue(s) being directly linked to the condensed aromatic system, it can be linked to the condensed aromatic system via intermediate residue groups, for example condensed rings, or other linker groups, e.g. (hetero-) alkyl or (hetero-) aryl groups.

Using compounds according to the invention, various EL devices can be constructed. The inventive compounds have the advantage that the core structure according to general formula I can be adapted by pre-selecting its substituents for specific EL functions, generating a compound with specific EL properties. From these compounds, layers in EL devices can be deposited from solution or by vapour deposition having pre-determined electrical and/or optical properties. Compounds comprising this core structure share the advantage of having high glass transition temperatures, a good solubility in organic solvents and, preferably, also a high long term stability.

### DETAILED DESCRIPTION OF THE INVENTION

The invention will now be described with reference to the figures, wherein

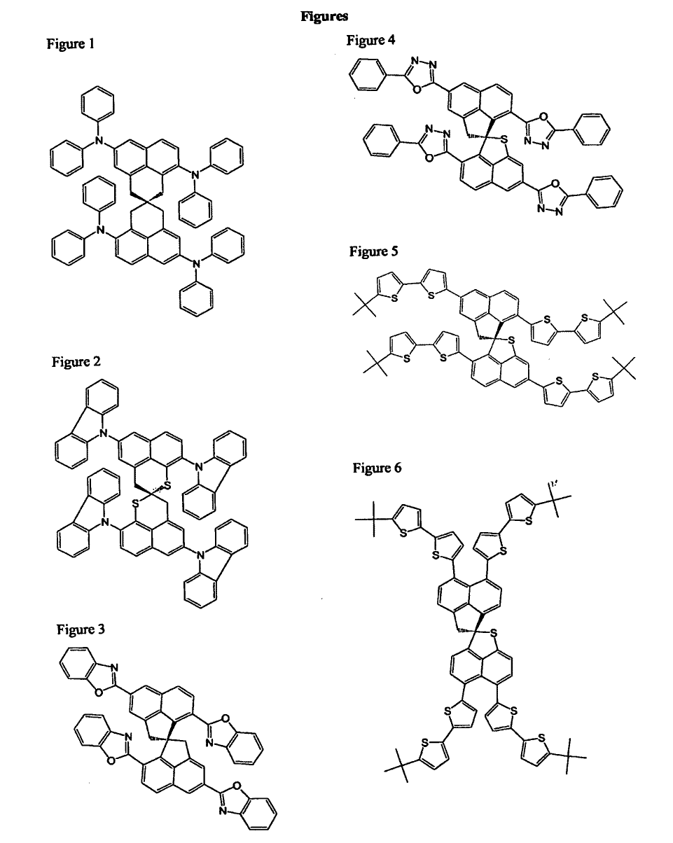

FIG. 1 describes a compound according to the invention having hole transporting properties,

FIG. 2 shows the structure of a compound according to the invention having hole transporting properties,

FIG. 3 shows a compound according to the invention having electron transporting properties,

FIG. 4 shows a compound according to the invention having electron transporting properties,

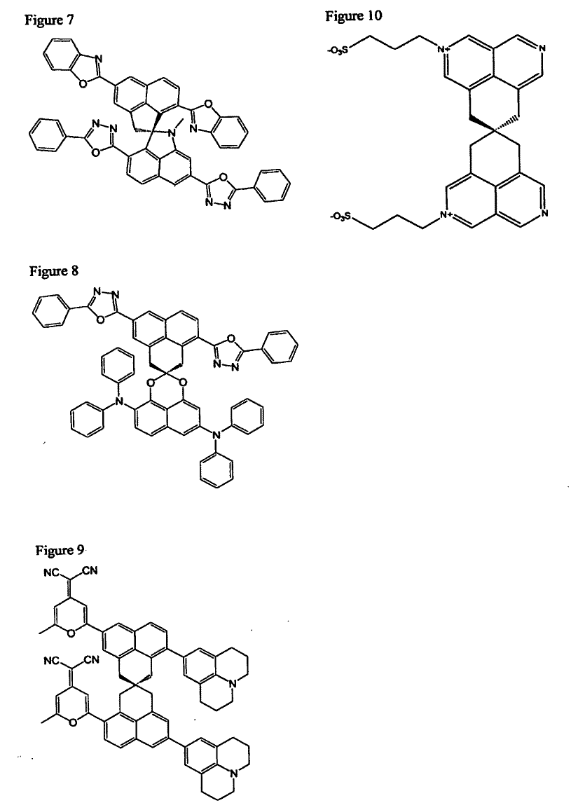

FIG. 5 shows a compound according to the invention having hole transporting properties,

FIG. 6 shows a compound according to the invention having hole transporting properties,

FIG. 7 shows a compound according to the invention having the property of electron transport,

FIG. 8 shows a compound according to the invention having combined properties of hole transport and electron transport,

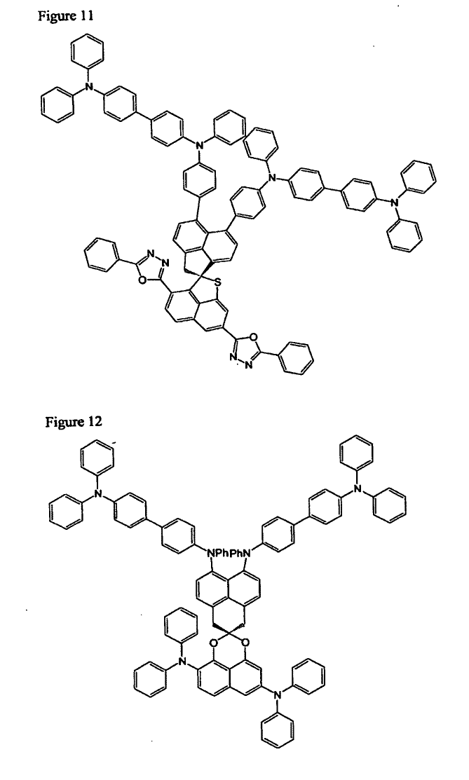

FIG. 9 shows a compound according to the invention having emitter properties,

FIG. 10 shows a compound according to the invention having emitter properties,

FIG. 11 shows a compound according to the invention having combined properties for hole and/or electron transport,

FIG. 12 shows a compound according to the invention having hole transporting properties,

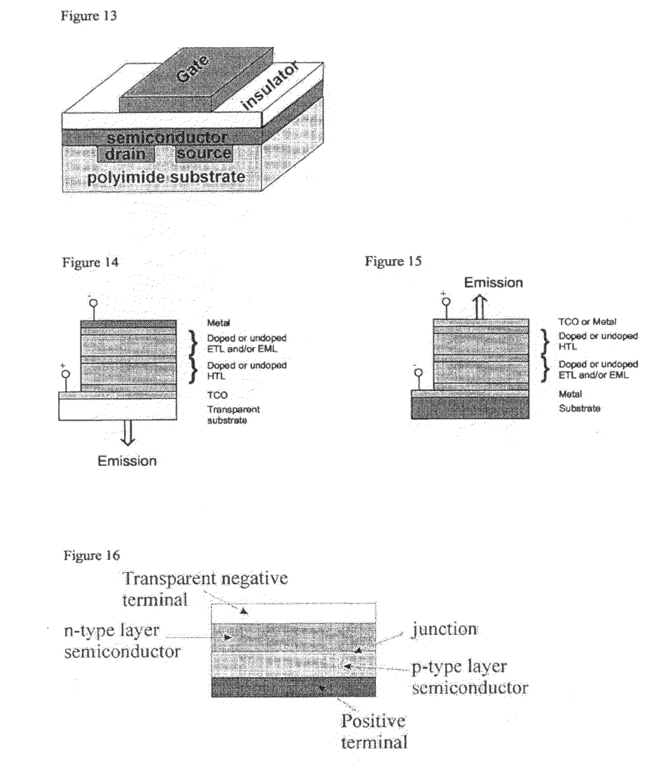

FIG. 13 schematically depicts an organic field electric transistor (OFET) in cross-section.

FIG. 14 schematically depicts an inverted OLED in cross-section,

FIG. 15 schematically depicts an OLED in cross-section, and

FIG. 16 schematically depicts a solar cell in cross-section.

The following examples depict some combinations of the inventive core structure with EL substituents. However, exchanges between the exemplary compounds in respect of the EL functional substituents as well as the structure of the core structure in respect of its embodiment as a four- or five-membered ring or a six-membered ring, independently, between each condensed aromatic system and the central atom Z, and embodiments comprising one or both of the condensed aromatic systems as part of higher anellated systems are comprised as embodiments of the invention.

As shown in the following examples, the substituent moieties need not be symmetrical to the central atom. The substituent moieties can comprise different EL functional groups or other residues in various positions of the condensed aromatic systems.

Synthesis of compounds according to the invention can be achieved according to known methods.

### EXAMPLE 1

### Hole Transport Material

As shown in FIG. 1, a transport material based on the core structure according to the invention may be formed by di-substituting each naphthyl group with two diphenylamino groups each. In this embodiment, the core structure according to the invention uses a carbon atom as the central atom Z. Intermediate residues (X, Y and V, W) linking each naphthyl group forming the condensed aromatic system with the central carbon atom are methylene groups, effectively forming a six-membered ring comprising the α and α′carbon atoms, respectively, of each naphthyl group, two intermediate methylene groups and the central carbon atom.

The naphthyl groups are each embodied without further condensed moieties and they are only substituted with EL functional groups in positions 2 and 6, providing the desired charge transporting properties.

### EXAMPLE 2

### Hole Transport Material

The compound of FIG. 2 shows a similar core structure as FIG. 1, having two sulfur atoms as intermediate residues to constitute six-membered rings each, formed between the α and α′ carbon atoms of the first and second naphthyl group, respectively, including the central carbon atom, and two sulfur atoms.

The naphthyl groups do not form part of a higher anellated aromatic system. The naphthyl groups are each substituted with two carbazole substituents in positions 2 and 6.

### EXAMPLE 3

### Electron Transport Material

As shown in FIG. 3, the core structure of this compound is formed of two opposite naphthyl groups, linked to the central carbon atom forming five-membered rings between the α and α′ carbon atoms of the naphthyl groups, respectively, a direct bond to the central carbon atom, the central carbon atom itself and an intermediate methylene group each. Both naphthyl groups are substituted with two moieties each to provide for the desired electronic properties, namely benzoxazole groups.

### EXAMPLE 4

### Electron Transport Material

The compound depicted in FIG. 4 shows the opposite naphthyl groups linked to the central carbon atom, each naphthyl group forming with its α and α′ carbon atoms, respectively, a five-membered ring with the central atom. One five-membered ring has a methylene group as the intermediate residue, whereas the opposite five-membered ring has a sulfur atom as the intermediate residue, with the other intermediate residue of each ring being formed by a chemical bond. The substituent groups to the naphthyl groups are 2-phenylbenzoxadiazole groups.

### EXAMPLE 5

### Hole Transport Material

The compound shown in FIG. 5 has an identical core structure to the compound of Example 4, however, the naphthyl groups are substituted with a chain of thienyl groups linked via their α carbon atoms, the terminal thienyl groups are substituted with tertiary butyl groups. This compound is suitable for forming a hole transporting layer in EL devices.

### EXAMPLE 6

### Hole Transport Material

The compound shown in FIG. 6 has a core structure identical to Examples 4 and 5. The two naphthyl groups are substituted on their respective delta carbon atoms. The substituent groups are α, α dithienyl groups with their terminal thienyl group substituted on its a carbon with a tertiary butyl group.

### EXAMPLE 7

### Electron Transport Material

The compound shown in FIG. 7 comprises a core structure comprising two opposite naphthyl groups, each forming a five-membered ring with the central carbon atom. One of the five-membered rings contains a methylene group as the intermediate residue, the opposite five-membered ring contains a nitrogen atom, substituted with a naphthyl group as the intermediate residue.

The naphthyl groups are substituted with moieties which confer electron transport properties, namely a 2-phenyloxadiazole substituent on one naphthyl group and a benzoxazole substituent on the opposite naphthyl group. The substituents to the naphthyl moieties are conjugated in positions 3 and 4 of the respective naphthyl groups.

### EXAMPLE 8

### Combined Hole and Electron Transport Material

The compound shown in FIG. 8 comprises the core structure having six-membered rings of connecting each naphthyl group to the central carbon atom, wherein the six-membered ring comprising the α carbon atoms of one naphthyl group comprises two methylene groups as intermediate residues, the six-membered ring linking the opposite naphthyl group to the same central carbon atom comprises oxygen as such intermediate residues.

The hole transport property is conferred by two diphenylamino substituents, the electron transport property by two 2-phenyloxadiazole substituents.

### EXAMPLE 9

### Emitter Compound

The compound shown in FIG. 9 has a core structure comprising two naphthyl groups connected to the central carbon atom via six-membered rings, each comprising two methylene groups as intermediate residues.

The naphthyl groups are substituted in position 3 with a dye acceptor moiety, and in position 7 with a dye donor moiety. As a result, a derivative of a merocyanine is formed, in this example corresponding to the known laser dye DCM2.

### EXAMPLE 10

### Emitter Compound

The compound shown in FIG. 10 gives an example for generating cyanine structures in core structure I. Two opposite [2,7]naphthyridine moieties having one quarternized nitrogen atom each are linked to the central carbon atom forming six-membered rings each with their two respective methylene groups as intermediate residues. The quartemizing substituent propylsulfonic acid renders the dye compound betainic. This compound is suitable as an emitter material in EL devices for blue wavelengths, e.g. as a dye component.

### EXAMPLE 11

### Hole Transporting Compound

The compound according to FIG. 11 comprises a core structure having two opposite naphthyl groups, each connected vis a five-membered ring to the central carbon atom, one five-membered ring having a methylene group as the intermediate residue, the opposite five-membered ring having a sulfur atom as the intermediate residue.

One naphthyl group is substituted in positions 1 and 8 with EL functional moieties, namely electron transporting substituent 2-phenylbenzoxadiazole. The opposite naphthyl group is substituted in positions 3 and 7 with diphenylamino substituents, conferring the property of hole transport.

### EXAMPLE 12

### Hole Transporting Compound

The compound shown in FIG. 12 comprises the core structure of two opposite naphthyl groups, each connected via a six-membered ring to the central carbon atom, one six-membered ring comprising methylene groups as the intermediate residues, the opposite six-membered ring comprising oxygen as intermediate residues.

For this compound, hole transporting moieties are present in positions 1 and 8 of one naphthyl group, and diphenylamino groups in positions 2 and 6 of the opposite naphthyl group. However, the positions of the substituent groups on the respective naphthyl groups as well as the substituent groups themselves can be exchanged from one naphthyl group to the other.

### EXAMPLE 13

### EL Devices

Compounds comprising the core structure according to the invention can be adapted to have pre-determined electrical and/or optical properties by selecting substituent groups conferring the desired EL properties. Accordingly, compounds according to the invention can be used to form layers in EL devices, wherein the layers require the respective EL properties of the compound. Exemplary EL devices are depicted in FIGS. 13 to 16, showing an OFET, an inverted OLED in cross-section, an OLED, and a solar cell.

1. Compound useful for electronic and/or electrooptic devices, characterized by a core structure of formula I: 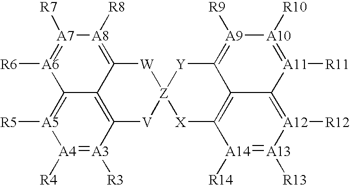
wherein V, W, X and Y are selected from at least divalent atoms or groups,
wherein at least one of V, W and X, Y, respectively, is no chemical bond but an at least divalent atom or group,
wherein R3 to R14 are independently selected from (hetero-) alkyls, (hetero-) aryls,
—NR′2, —OR′, —SR′, —CN, —F, —CF₃, with R′ independently selected from substituted or unsubstituted (hetero-)alkyl, (hetero-)aryl or hydrogen, and electrooptically functional groups,
wherein A3 to A14 are independently carbon or nitrogen atoms and
wherein Z is a central tetraedric atom.

2. Compound according to claim 1, characterized in that V, W, X and Y are selected from the group comprising —S—, —O—, a carbonyl group, —SO₂—, and di-substituted silicon, —CRR—, and a chemical bond, with R any (hetero-) alkyl or (hetero-) aryl or hydrogen.

3. Compound according to claim 1 or 2, characterized in that A3 to A14 are carbon atoms.

4. Compound according to one of the preceding claims, characterized in that two or more of R3 to R14 are condensed arenyl groups or are groups forming a higher condensed analog of the core structure of formula I.

5. Compound according to one of the preceding claims, characterized in that central tetraedric atom Z is selected from the group comprising silicon, germanium and carbon.

6. Compound according to one of the preceding claims, characterized in that least one of R3 to R14 is selected from hole transporting residues, electron transporting residues and emitter residues.

7. Compound according to claim 6, characterized in that a hole transporting moiety is selected from the group of tris-[(N,N-diaryl)amino-triphenylamines like 4,4′,4″-tris[(N-(1-naphthyl)-N-phenyl-amino-triphenylamine] (1-TNATA) and its derivatives, 4,4′,4″-tris[(N-(2-naphthyl)-N-phenyl amino)-triphenylamine] (2-TNATA) or 4,4′,4″-tris[N-(3-methylphenyl)-N-phenyl-amino)-triphenylamine] (m-TDATA) and its derivatives, 4,4′,4″-tris(carbazole-9-yl)triphenylamines; N,N,N′,N′-tetra arylbenzidines as N,N,N′,N′-tetraphenylbenzidine and its derivatives, N,N′-bis(1-naphthyl)-N,N′-diphenyl-benzidine (α-NPD), N,N′-di(naphthalene-2-yl)-N,N′-diphenyl-benzidine (β-NPD), 4,4′-bis(carbazole-9-yl)biphenyl (CBP) and its derivatives, and their heteroatom substituted analogs (e.g. thienyl-, selenyl-, furanyl-derivatives); 4,4′-bis(2,2′-diphenylvinyl)-1,1′-biphenyl (DPVBI); triarylamines and their derivatives, 4,4′-bis(N,N-diarylamino)-terphenyls, 4,4′-bis(N,N-diarylamino)-quarterphenyls and their homologs and derivatives.

8. Compound according to claim 6 or 7, characterized in that an electron transporting moiety is selected from the group of 4,7-diphenyl-1,10-phenanthroline (Bphen) and derivatives thereof as 2,9-dimethyl-4,7-diphenyl-1,10-phenanthroline (BCP), 2,5-diaryloxadiazoles and derivatives thereof as 2-(p-tert-butylpheny1)-5-(p-biphenyl)-oxadiazole (PBD), oligo-(benzoxadiazol-2yl)-arenes and derivatives thereof as bis-2,5-(5-tert. -butyl-benzoxadiazol-2-yl)-thiophene (BBOT), 1,3-bis[5-(aryl)-1,3,4-oxadiazol-2yl]benzenes and derivatives thereof as 1,3-bis[5-(p-tert.-butylphenyl)-1,3,4-oxadiazol-2yl]benzene (OXD-7), 2,5-diaryltriazoles and derivatives like 2-(p-tert.-butylphenyl)-5-(p-biphenyl)-triazole (TAZ).

9. Compound according to claims 6 to 8, characterized in that an emitter moiety is selected from the group comprising coumarins, rhodamines, merocyanines, cyanines and oxonoles.

10. Compound according to claims 6 to 9, characterized in that an emitter moiety is formed by two or more of R3 to R14 that are condensed arenyl groups.

11. Compound useful for electronic and/or electrooptic devices, represented by one of the following formulae:

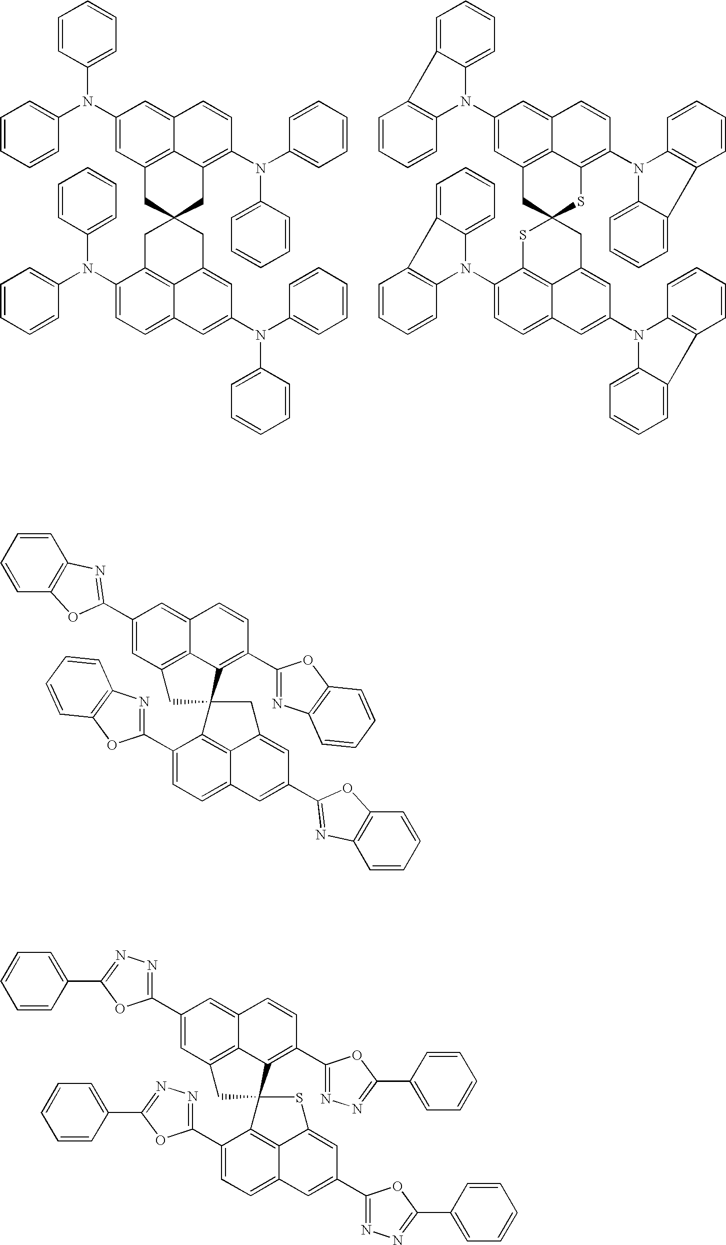

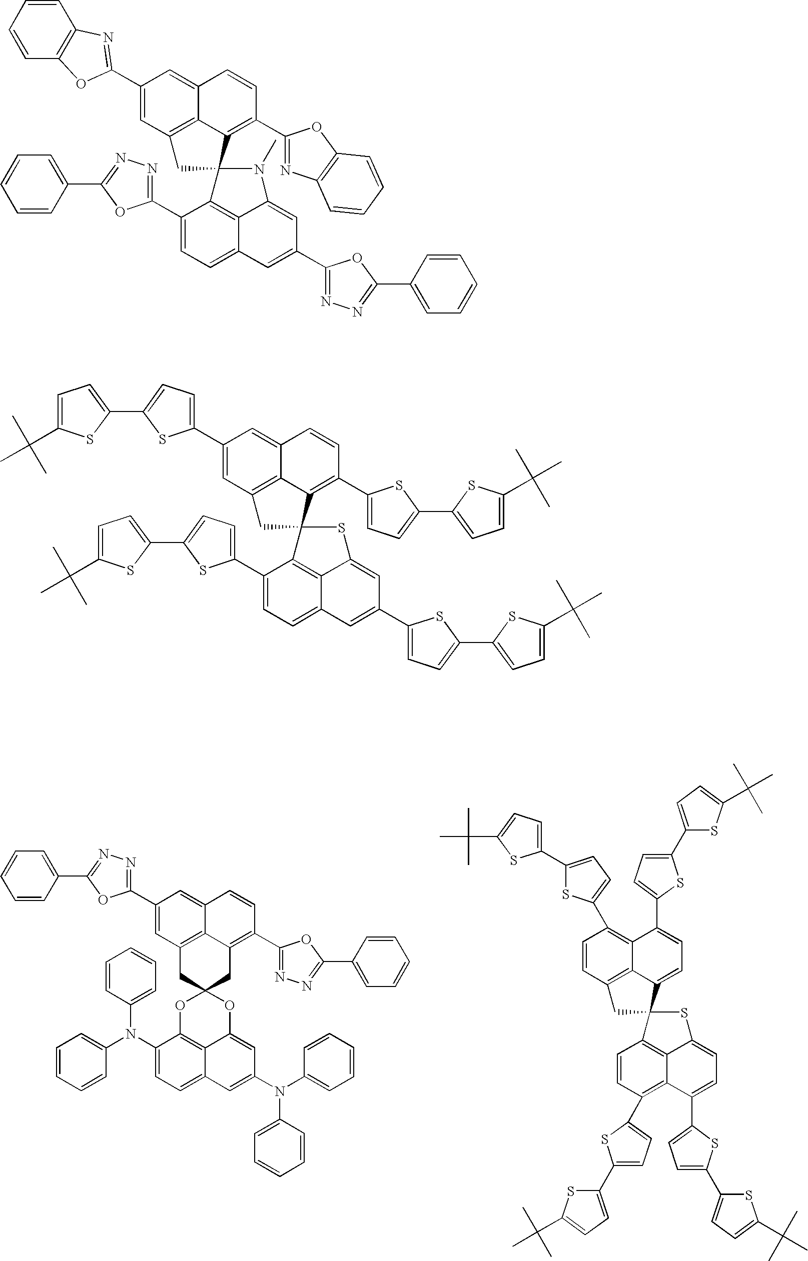

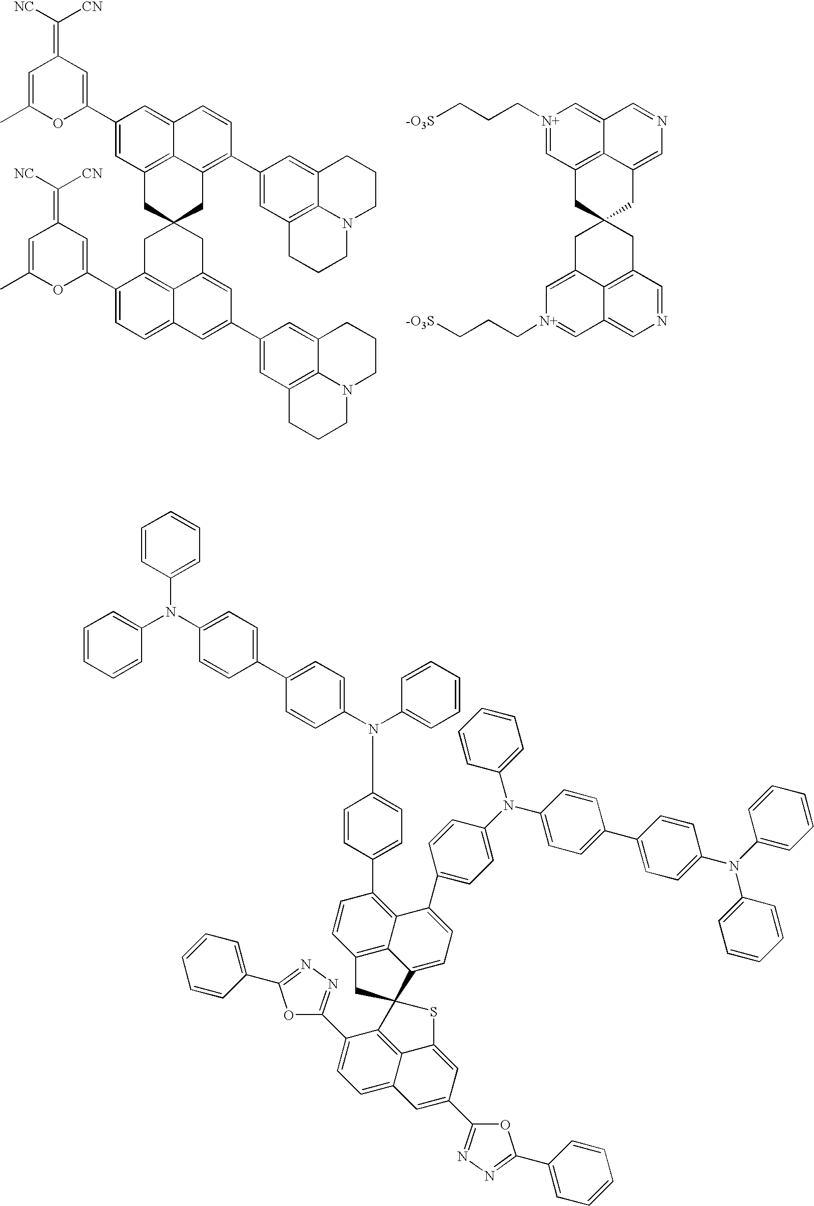

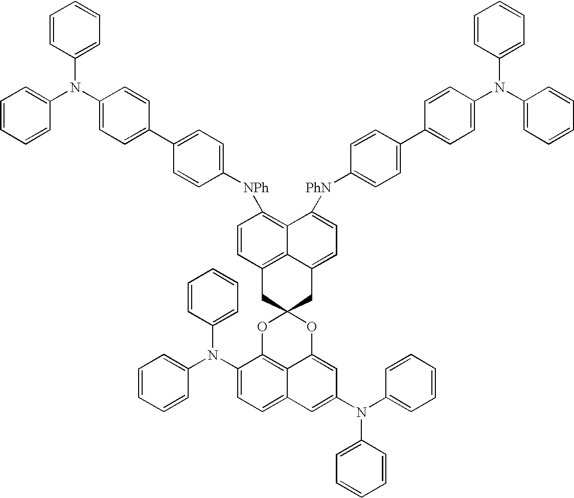

12. Compound according to one of the preceding claims, characterized in that the compound is a hole transporter, electron transporter and/or emitter.

13. Process for synthesis of compounds useful for electroluminescent devices, characterized by generating a compound according to one of the preceding claims.

14. Electronic, electrooptic or electroluminescent device, characterized by comprising a compound according to claims 1 to 12.
